(19) United States
(12) Patent Application Publication
(10) Pub. No.: US 2014/0363529 A1
(43) Pub. Date: Dec. 11, 2014

(54) COMPOUNDS INFLUENCING FATTY ACID UPTAKE AND METABOLISM AND METHODS OF ISOLATING FROM COCOA PRODUCTS

(71) Applicants: W. Jeffrey HURST, Mt. Gretna, PA (US); David A. STUART, Hershey, PA (US); Joshua D. LAMBERT, State College, PA (US); Yeyi GU, State College, PA (US)

(72) Inventors: W. Jeffrey HURST, Mt. Gretna, PA (US); David A. STUART, Hershey, PA (US); Joshua D. LAMBERT, State College, PA (US); Yeyi GU, State College, PA (US)

(73) Assignee: The Hershey Company, Hershey, PA (US)

(21) Appl. No.: 13/913,121

(22) Filed: Jun. 7, 2013

(51) Int. Cl. A61K 31/353; A61K 45/06; A61K 36/185

(52) U.S. Cl. CPC A61K 31/353; A61K 36/185; A61K 45/06
USPC 424/769; 514/456; 549/406; 514/263.34; 435/375

(57) ABSTRACT

The invention provides compounds and plant extract compositions that inhibit pancreatic enzymes, such as lipases and amylases, and most particularly pancreatic lipase and phospholipase A₂ (PLA2), and COX-2 enzyme, and improve the inflammatory state or response conditions in animals. The compounds and plant extracts can be used in methods and administration regimens to treat animals for obesity-related conditions, diabetes and related conditions, metabolic syndrome, metabolic endotoxemia, and inflammatory conditions. The compounds and plant extracts can also be used to produce comestible compositions to be incorporated into a normal diet to improve health or prevent or reduce the uptake of free fatty acids during digestion or the production of inflammatory eicosanoids or cytokines. The inhibitor compounds and compositions include cocoa-derived polymers of epicatechin, such as epicatechin-rich polymers of 2 units through polymers of 14 units and combinations of them.

### REFERENCE TO RELATED APPLICATIONS

This application claims priority benefit of PCT application PCT/US2011/063806, filed Dec. 7, 2011, and U.S. Provisional Application 61/420,701, filed Dec. 7, 2010, and the entire contents of each are hereby incorporated by reference.

### FIELD OF THE INVENTION AND INTRODUCTION

The invention relates to enzyme inhibitors and biologically active polymers present in plants, especially Theobroma cacao, that can be used to improve health or reduce fatty acid uptake during digestion. In general, the compounds and plant extract compositions inhibit pancreatic enzymes, such as lipases and amylases, or effect the production of inflammatory eicosanoids or cytokines. In certain aspects of the invention, the compounds and compositions are particularly useful for inhibiting pancreatic lipase and phospholipase A₂ (PLA2) enzymes in a dose-dependent manner, and can be used in therapeutic or prophylactic treatments in animals and humans. The compounds and plant extracts can be used in methods and administration regimens to treat animals for obesity-related conditions, diabetes and related conditions, metabolic syndrome, and inflammatory conditions. The compounds and plant extracts can also be used to produce comestible compositions and products to be incorporated into a normal diet to improve health or prevent or reduce the uptake of free fatty acids during digestion. The inhibitor compounds and compositions include cocoa-derived polymers of epicatechin and catechin, such as polymers of 2 units through polymers of 14 units (DP=2-14), or higher polymers, and various combinations of these polymers, and in particular the purified DP5, DP7, DP8, and purified DP10 polymers, or purified extracts containing one of DP5 to DP14 or combinations of them. In other aspects, the cocoa-derived polymers reduce the production of mammalian eicosanoids, such as prostaglandin (PG)E₂, in a dose-dependent manner. And in yet another aspect, the cocoa-derived polymers and compositions of them inhibit the production of inflammatory cytokines interleukin 6 (IL-6) and tumor necrosis factor (TNF) in stimulated mammalian cells, improve the inflammatory state of animals, and can be part of treatments for chronic inflammatory diseases, liver diseases, diabetes, and cardiovascular diseases.

### RELEVANCE OF THE INVENTION AND DESCRIPTION OF RELATED ART

There are likely hundreds of separate compounds that can be isolated from Theobroma cacao beans. More and more evidence shows the health benefits of many of these compounds, especially cocoa antioxidants or cocoa flavanol compounds. Most of the evidence relates to cellular studies or conditions where cells of an animal are directly treated by these compounds. These studies attempt to simulate conditions found after the absorption of cocoa through the gut.

The invention, in one aspect, provides methods to beneficially inhibit pancreatic enzymes prior to the absorption of fatty acids during normal, mammalian digestion. These methods can be incorporated into treatment regimens or administration routines to adjust or alter the diet of subjects in need of weight loss, dietary changes due to metabolic syndrome or diabetes, or other health conditions. In addition, food products or other comestible or ingestible products can incorporate effective amounts of the cocoa-derived compounds noted here. In other aspects, the invention relates to compositions of biologically active and purified epicatechin polymers, or combinations of certain cocoa-derived polymers, and their use in preventing inflammatory conditions in humans and mammals. These uses include methods to reduce the production or secretion of inflammatory eicosanoids and cytokines, as well as methods to bind the PLA2 enzyme with cocoa-derived and epicatechin polymers (PC).

### BRIEF DESCRIPTION OF THE DRAWINGS

FIG. 1(a) shows a graph of the in vitro α-amylase enzyme activity, and its inhibition as a percent of control, over various concentrations of cocoa-derived compositions. Extracts from regular or natural cocoa powder, unfermented or lavado cocoa, and alkaline treated or dutched cocoa powder are shown. The lavado-sourced compositions exhibit the highest levels of inhibitory activity.

FIG. 1(b) shows a graph of the in vitro pancreatic lipase enzyme activity, and its inhibition as a percent of control, over the same cocoa compositions and concentrations shown in FIG. 1(a).

FIG. 1(c) shows a graph of the in vitro phospholipase A2 enzyme activity, and its inhibition as a percent of control, over the same cocoa compositions and concentrations shown in FIG. 1(a) and FIG. 1(b). Both of the compositions derived from lavado and regular cocoa show a significant inhibition of phospholipase A2 (PLA2) enzyme.

FIGS. 2(a) through 2(c) show graphs of the inhibition of the three enzymes tested in FIGS. 1(a) to 1(c) when a specific subset of epicatechin polymers are administered to the enzyme. Polymer compositions were separated by size and degree of polymerization, with DP=2 referring to a degree of polymerization of two (2), and DP=10 a degree of polymerization of ten (10). FIG. 2(a) shows the inhibition of α-amylase to each of the polymer compositions. EC is control epicatechin monomer. FIG. 2(b) shows the inhibition of pancreatic lipase to each of the polymer compositions.

FIG. 2(c) shows the inhibition of PLA2 to each of the polymer compositions. As shown in FIG. 2(c), the polymer compositions of DP 5 to DP 10 exhibit a high level of enzyme inhibiting activity at very low concentrations.

FIG. 3 shows the same results as in FIG. 2(b) but with additional data showing the inhibition of pancreatic lipase by the drug Orlistat for comparison.

FIG. 4 shows the half of maximum Inhibitory Concentration (IC50) of the polymer compositions for each enzyme tested in FIGS. 1-3. Based on the dose-response curves for the purified polymer compositions, DP appears to be an important factor determining the potency of the compound or composition. By regression analysis, there is a strong inverse relationship between Log IC50 and DP (R2>0.93, FIG. 4). A similar analysis comparing Log IC50 and hydrophobicity (Log P) shows no significant correlation (data not shown).

FIGS. 5(a) to 5(c) show an analysis of the kinetics for pancreatic lipase (PL) and FIGS. 6(a) to 6(c) for PLA2, as both of these enzymes are more sensitive to inhibition by cocoa extracts and polymer compositions. The procyanidin pentamer (DP 5) and decamer (DP 10) as well as the regular cocoa extract are used as test inhibitors. All three test substances reduced the Vmax and increased Km of PL (FIG. 5) and as shown in the Table of FIG. 7. These results suggest a mixed-type inhibition with respect to substrate concentration. On the other hand, Michealis-Menten plots of PLA2 inhibition by the pentamer and decamer compositions showed non-competitive inhibition with respect to substrate concentration (FIGS. 6(a) to (c) and the Table of FIG. 8). By contrast, the regular cocoa extract demonstrated a competitive mode of inhibition against PLA2 with respect to substrate concentration (Table of FIG. 8)

FIG. 9 is a Table showing the summary of enzyme inhibition numbers for each of the polymer compositions DP 2 to DP 10 as well as epicatechin (EC) monomer.

FIGS. 10, 11 and 12 show the inhibition of a-amylase, pancreatic lipase, and phospholipase A2 by procyanidin polymers derived from apple. The data include the use of polymers DP 10 to DP 13. As is the case for cocoa-derived compounds and compositions, these compounds are strong inhibitors of PLA2 activity.

FIG. 13 shows the effect of a high molecular weight polymer mixture (degree of polymerization 7 or greater; DP7+Mix) on the production of the inflammatory eicosanoid prostaglandin (PG)E₂ by lipopolysaccharide (LPS, 1 μg/mL)-stimulated RAW264.7 macrophage cells. Three treatment regimens can be tested: (a) Pre-treatment with DP7+ for 6 h then stimulation with LPS for 6 h (Pre-cocoa); Pre-stimulation with LPS for 6 h then treatment with DP7+ for 6 h (Pre-LPS); and Co-treatment with DP7+ and LPS for 12 h (Cotreat). All three treatment regimens reduced the production of PGE₂ compared to control (0) and in a dose-dependent manner.

FIG. 14 shows the inhibitory effect of the DP7+ mixture of cocoa-derived polymers on purified cyclooxygenase 2 (COX-2) enzyme activity. Mean inhibitory concentration (IC₅₀) is 58 ug/ml.

FIG. 15 graph (A)(left) shows the inhibition of inflammatory cytokine (IL-6 and TNFα) production in LPS stimulated macrophages by administration of purified polymer DP=8. FIG. 15 graph (B)(right) shows the inhibition of PGE₂ production in LPS stimulated macrophages by purified DP=8 polymer. Macrophage cell cultures (RAW264.7) are pre-treated with DP=8 octamer for 10 h and then stimulated with LPS for 12 h and assayed.

FIG. 16 depicts the binding interactions of PLA₂ enzyme. Panel A shows ball-and-stick substrate bound in tunnel region of space-filling model of PLA₂ enzyme. Panel B depicts DP=2 polymer interacting with secondary and tertiary structures of PLA₂ enzyme, including binding to helices that form tunnel walls as shown in Panel A. Panel C depicts DP=7 binding to secondary and tertiary structures of PLA₂ enzyme. In Panel C, additional binding sites of DP=7 polymer exhibits potential for increased potency as inhibitory molecule.

FIG. 17A depicts the mouse adipose tissue (AT) arachidonic acid levels measured after treatment with low fat (LF), high fat (HF), and high fat diet supplemented with cocoa powder (HFC). FIG. 17B depicts the correlation of this data with the adiposity of the animals.

FIG. 18 A-D shows the effect of cocoa supplementation on protein expression of various markers for inflammatory responses (AdPla—adipose tissue phospholipase A2; Cox-2 and 5-LOX involved with eicosanoids pathways, and master inflammatory marker NF-κB p65). Gapdh is used as background control for cell proteins and histone H3 is used as background control for nucleus proteins.

FIG. 19 shows the plasma endotoxins levels measured in mice treated with HF, LF, and HFC diets as noted above.

FIG. 20 A-B depicts the plasma GLP-2 levels present in mice treated with LF, HF, and HFC diets as noted above. In panel B, the data is correlated with the GLP-2 levels of the animals.

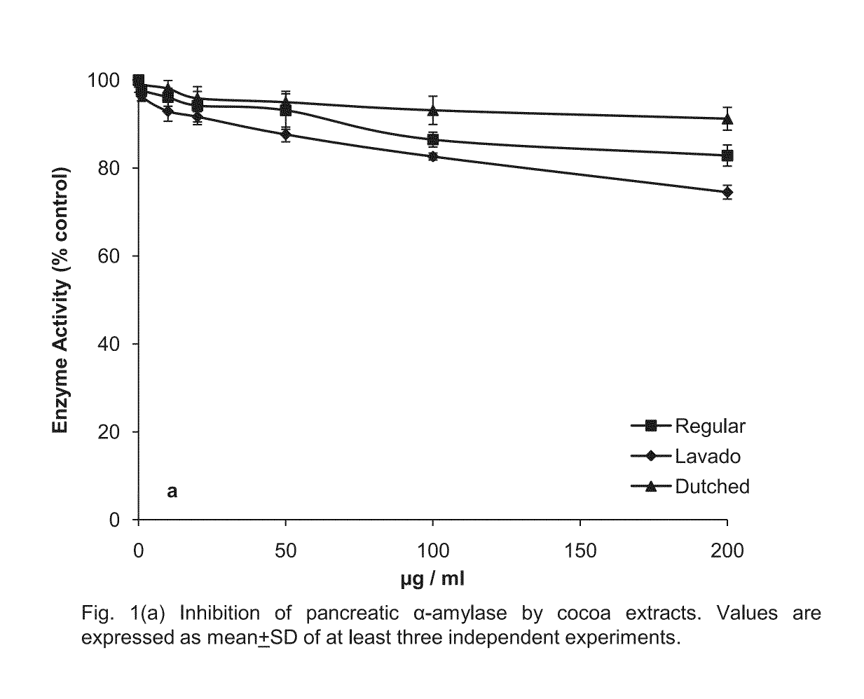

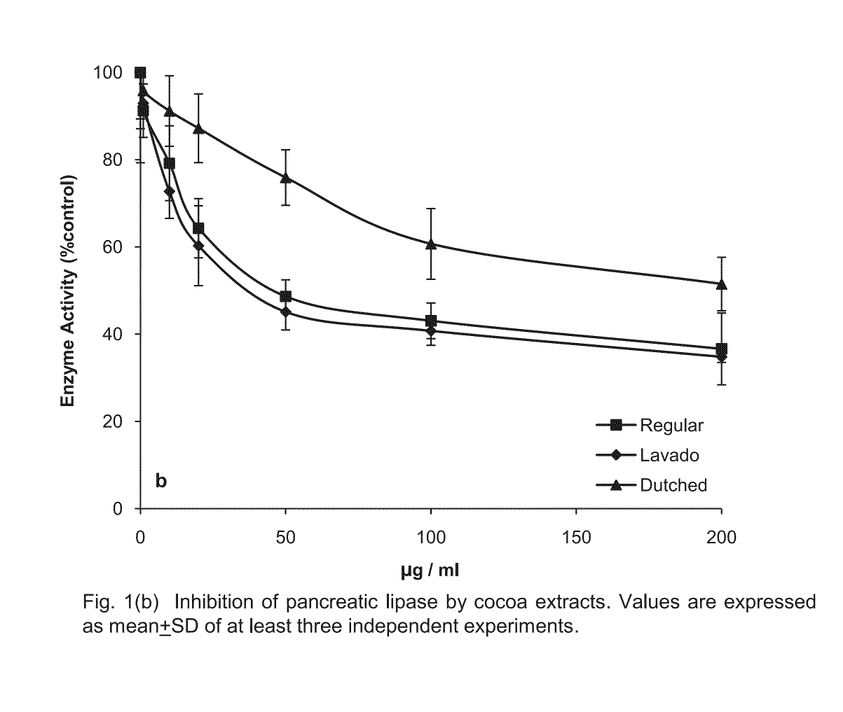

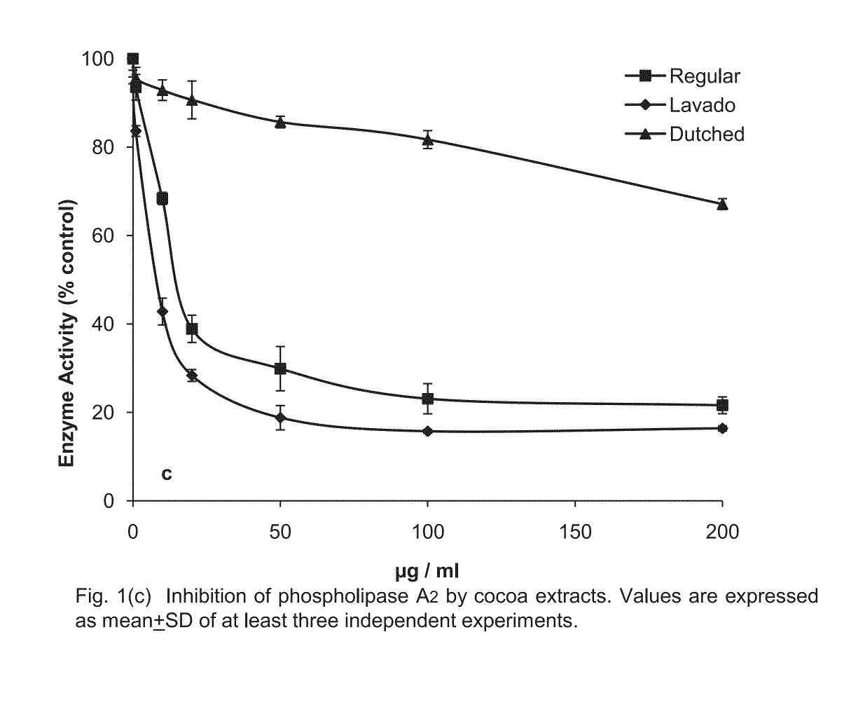

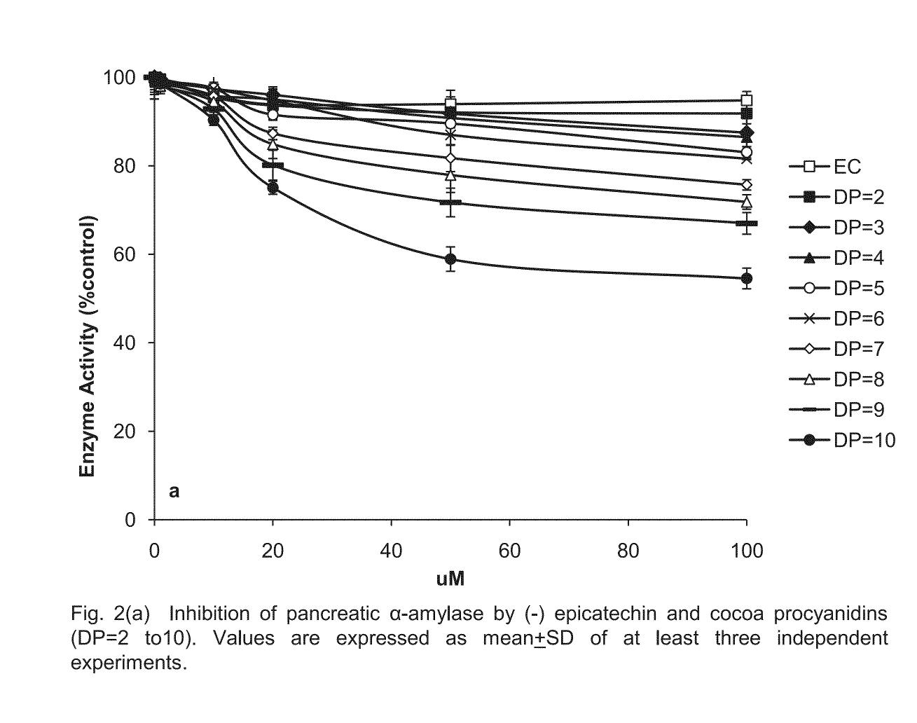

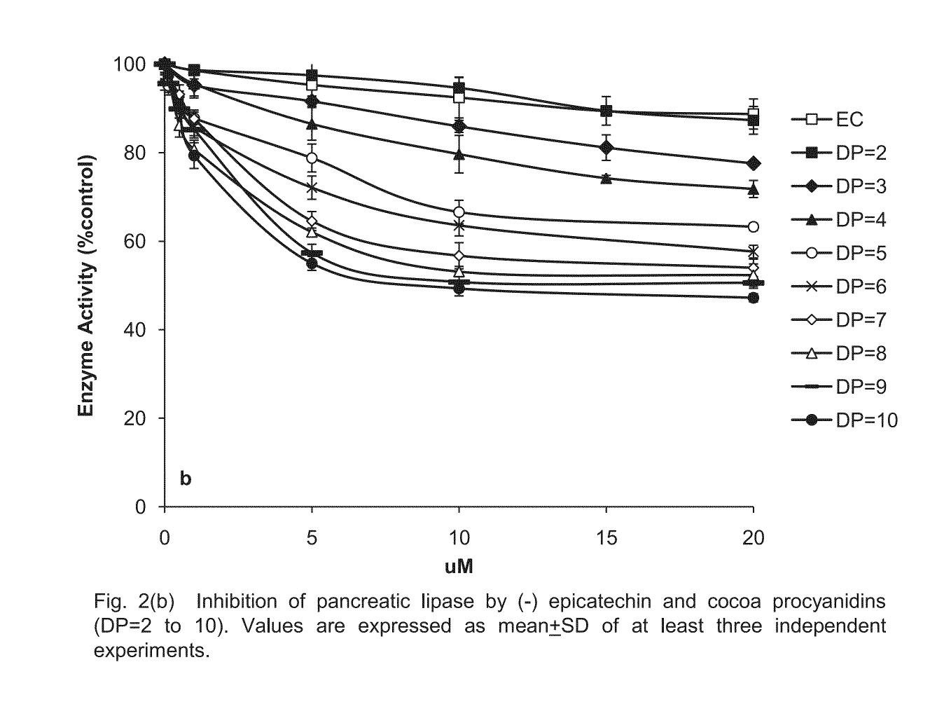

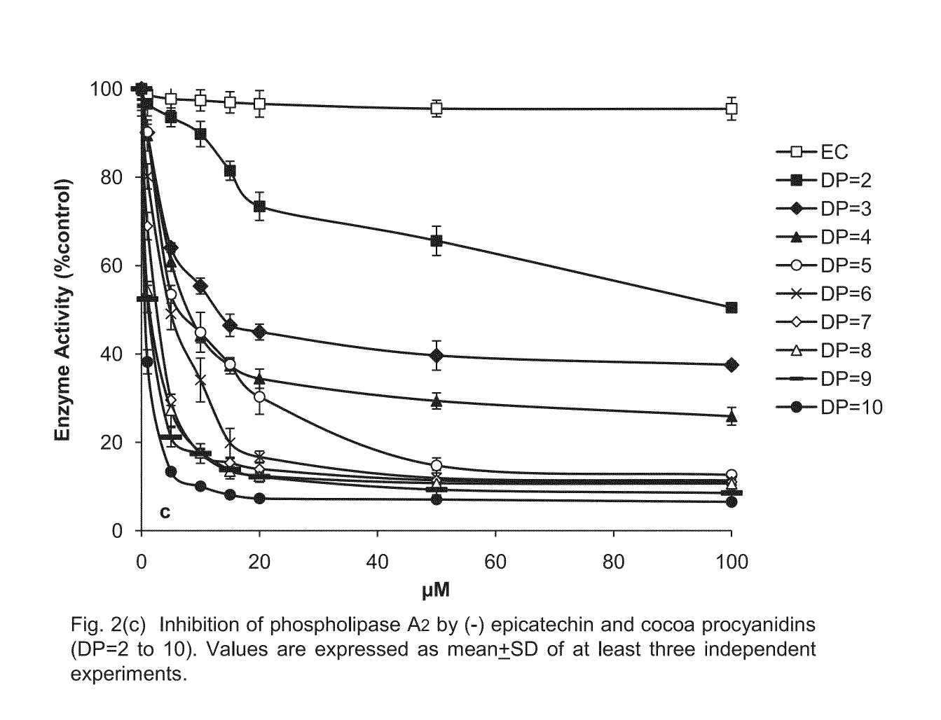

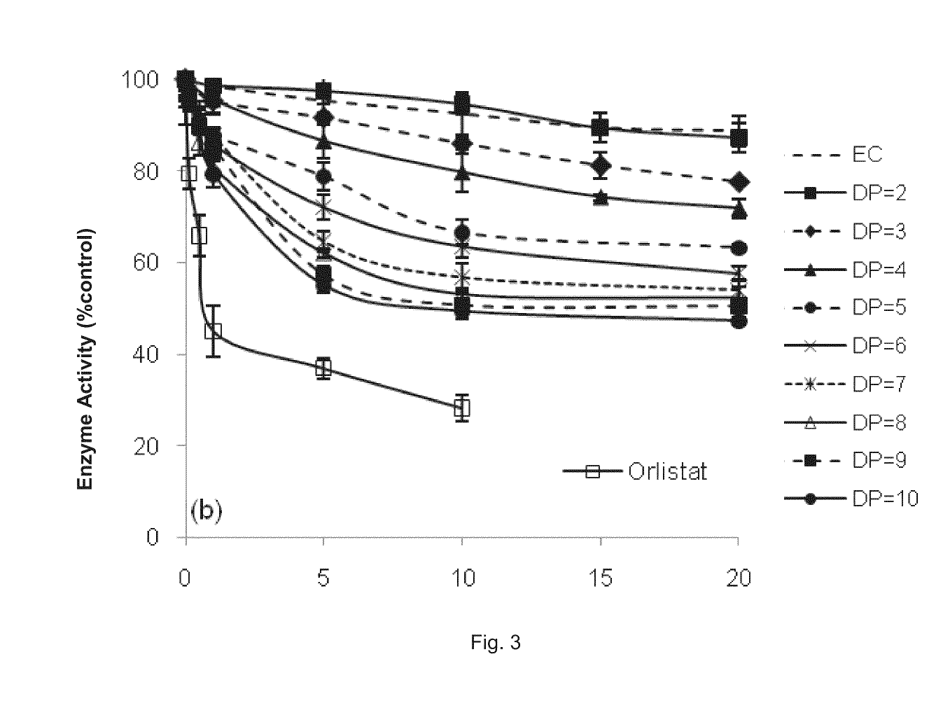

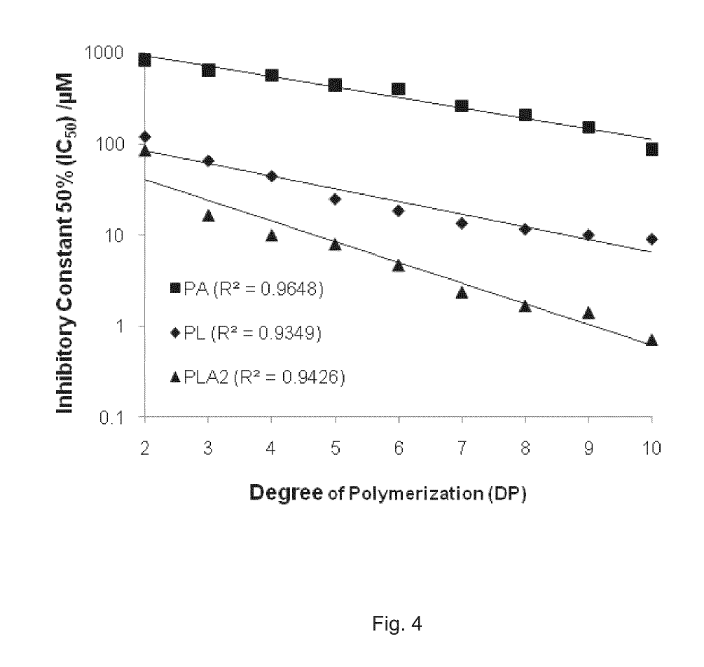

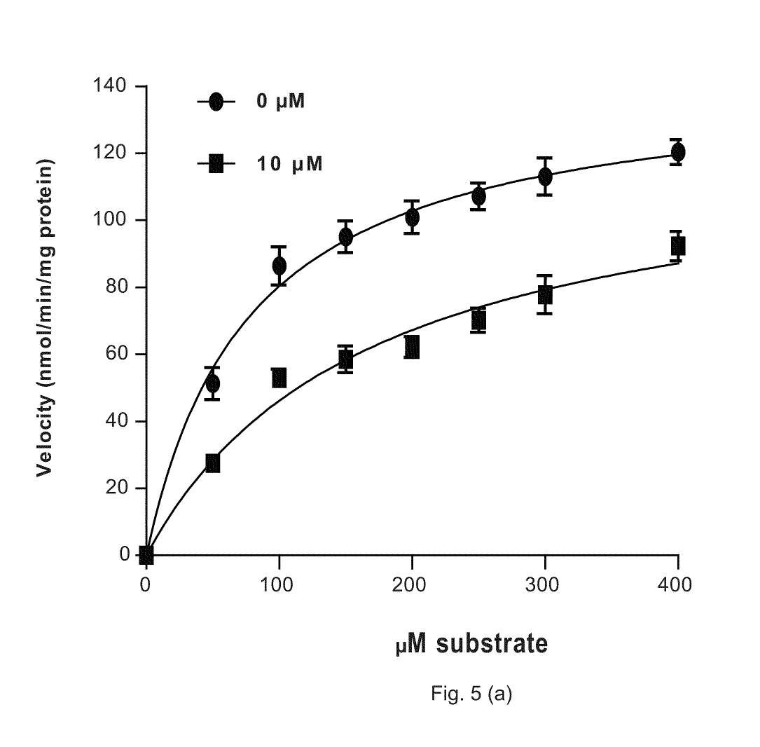

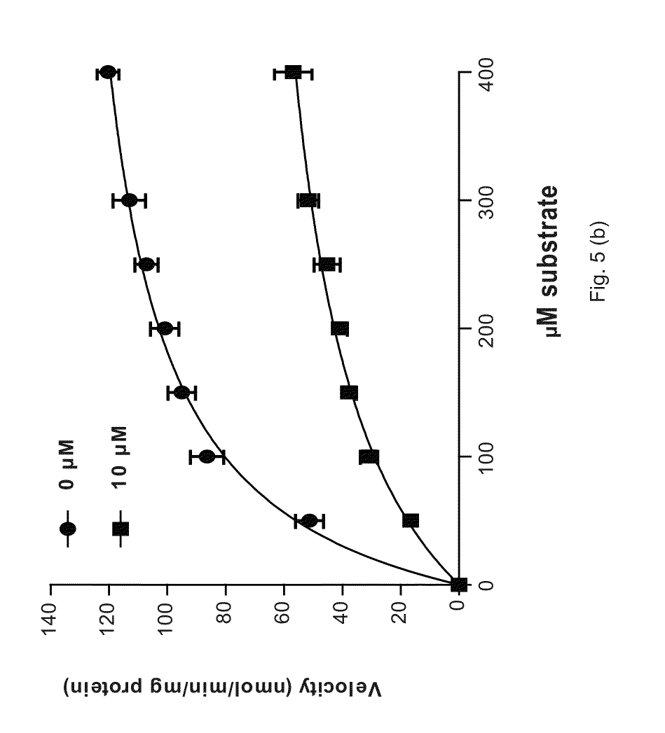

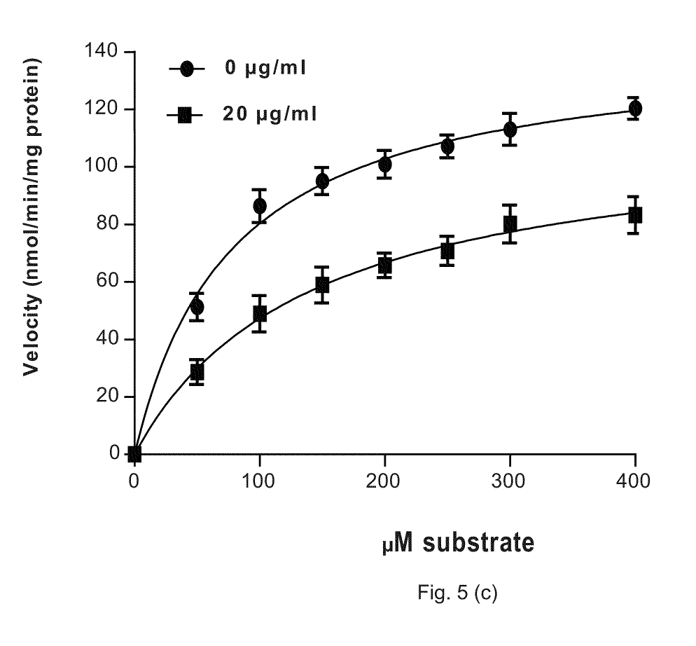

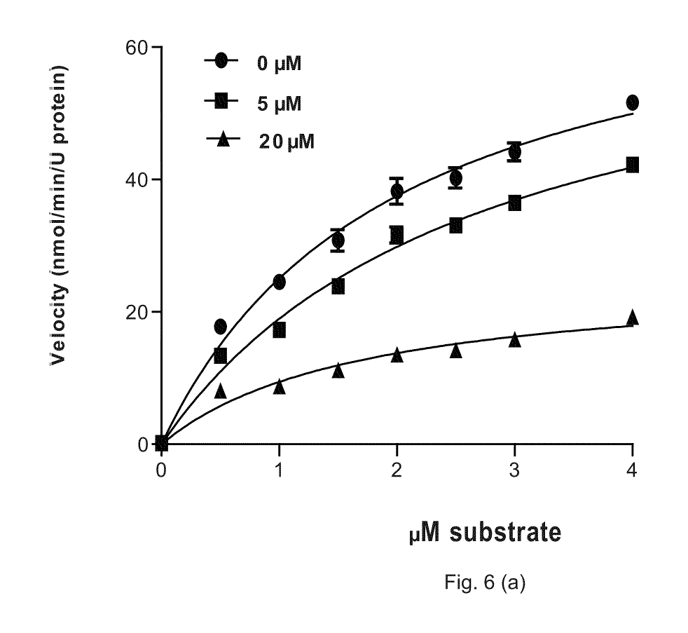

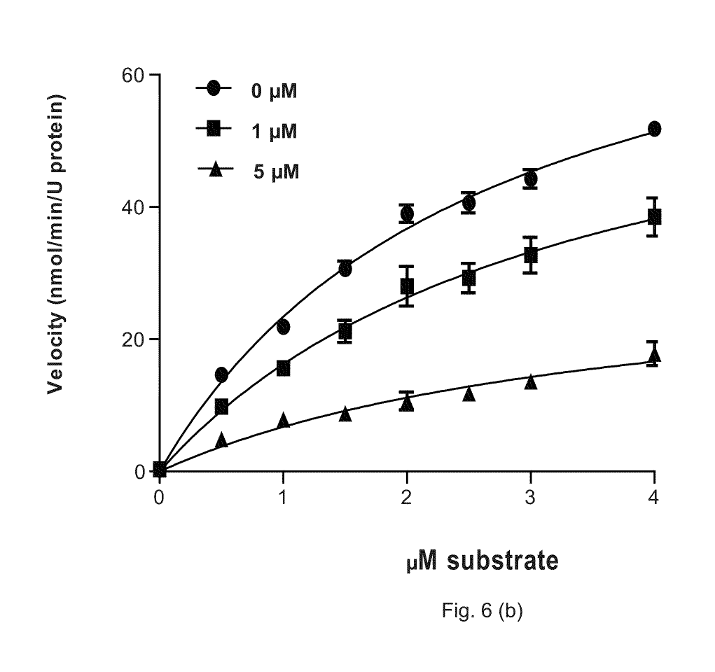

### SUMMARY OF THE INVENTION

The invention, in one aspect, satisfies a need for products and methods to inhibit fatty acid uptake and metabolism in mammalian subjects. Previously, no report discussed cocoa products, cocoa powders, or chocolate products as having a direct impact on fatty acid uptake or metabolism. The invention described here explains, at least in part, how the use of a cocoa powder-containing beverage or food composition can lead to a slightly smaller physical circumference in adults compared to the same adults on a controlled, placebo treatment (Fulgoni V., Fulgoni, S. & Bodor, A. (2009, April), Association of Candy Consumption with Physiological Parameters in Participants from the National Health and Nutrition Examination Survey (1999-2004); Poster session presented at the annual meeting of Experimental Biology, New Orleans, La.)). It has also been noted that despite having relatively high levels of saturated fat, chocolate does not adversely impact the level of LDL-cholesterol (Ding, E. L. et al., 2006, Chocolate and prevention of cardiovascular disease: A systematic review; Nutrition and Metabolism 3: 2-13) and evidence suggests that cocoa powder may even reduce levels of LDL-cholesterol in short-term studies (Jia, L et al., 2010, Short-term effects of cocoa product consumption on lipid profile: A meta-analysis of randomized controlled trials; Am. J. Clin. Nutr. 92:218-25). In combination with other known compounds found in cocoa, the new cocoa products, chocolates, and food ingredients and products advantageously provided by the invention create new possibilities for producing or supplementing foods with beneficial levels of natural cocoa-derived polymers and epicatechin polymers.

In another aspect, the invention involves the isolation and use of polymer compounds and polymer compositions from plants that specifically or non-competitively inhibit PLA2 enzyme activity. Thus, the polymer compositions, epicatechin-rich polymers, and cocoa-derived polymers and purified polymers and combinations can be used to effect levels of a variety of inflammation-related and immune response-related eicosanoid compounds, such as prostaglandins and leukotrienes, as well as effect the pathways and feedback mechanisms in their biosynthetic pathways. In addition, the data and examples here show how cocoa powder and cocoa procyanidins (b-type procyanidins) can in particular effect the COX-2 pathway and thus the prostaglandin cascade in animals, providing a basis for treatments with cocoa powder and cocoa procyanidins that improve the inflammatory profile and animals and thus improve health. In a related manner, the data and examples show that cocoa powder and cocoa procyanidin administration to animals improves the gut barrier function leading to improvements in endotoxin levels of obese animals or animals with compromised gut barrier function.

In another aspect, the invention includes polymer compositions and their use, for example in inhibiting PLA2 enzyme activity. In particular and preferably, parts and beans from Theobroma cacao can be used. Samples, mixtures and extracts derived from Theobroma cacao seeds (cocoa beans) contain especially high levels of epicatechin compounds, on the order of 30:1 epicatechin compared to catechin. In most other plants, this ratio is closer to 1:1. As used in this specification, the term “cocoa-derived epicatechin” polymer or composition or sample may refer to a sample or composition containing some level of other compounds within the polymer, such as catechin. The cocoa-derived epicatechin polymers are thus epicatechin-rich but may not be exclusively composed of epicatechin monomer units. As used herein, “epicatechin-rich polymers” refers to polymer compositions where on average the monomer units are predominantly epicatechin compounds, such as more than 70% or more than 80% or more than 90% or more than 95% epicatechin, or more, on average by weight. The purified samples referred to in this application refer to polymers substantially purified from polymers of another size, meaning the specific polymer is present in greater than 60%, or 70% or 80% or 90% or 95% of all polymers present. Preferably, the compositions of the invention will be derived from a cocoa source, most preferably a cocoa bean or the nib of a cocoa bean, and will predominantly contain polymers composed of epicatechin, in the ratio of approximately 30:1 over other related compounds that can be combined into a procyanidin polymer in plants. However, other plant materials can also be used, such as apple and sorghum, or other plant sources high in type-B procyanidin polymers of epicatechin. Accordingly, various polymer compounds as well as combinations of polymer compounds, combinations with other enzyme inhibitors, and compositions for oral administration, are specifically included in the invention.

In another aspect, the invention relates to methods for treating animal or human subjects in order to reduce the effectiveness of fatty acid digestion and thus reduce the uptake of fatty acids and/or triglycerides during digestion, or inhibit PLA₂ enzyme activity in the subject. The inhibitory compounds and compositions are especially useful in treating obesity, obesity-related disorders, diabetes, and diseases and conditions associated with inflammatory pathways and/or conditions where reduced eicosanoid or cytokine production could be beneficial. In a more general sense, the compounds and compositions can be used to alter fat or fatty acid metabolism in an animal or human. Similarly, the compounds and compositions are especially useful in treating liver disease, diseases associated with metabolic endotoxemia, diabetes, and cardiovascular disease.

In yet another aspect, the invention relates to the use of the compounds and compositions to prepare or manufacture a food product or orally-administered medicament or comestible composition. In another aspect, systemic or injectable compositions are possible, especially with purified polymer compounds and extracts that non-competitively inhibit PLA2 enzyme, an enzyme that can play an important role in inflammatory, cardiovascular, and nervous system disorders. Thus, the purified plant compounds and compositions can be used as a pharmaceutical for human treatment where the PLA2 enzyme is involved in the metabolism or catabolism of products that impact a disease condition.

In yet another aspect, the invention includes combinations of the enzyme inhibitor compounds and compositions with other enzyme inhibitors. For example, one or more of the inhibitors tetrahydrolipstatin or orlistat, phaseolamine, cetilistat, crocetin, lipstatin, vibralactone, and green tea catechins can be combined with the non-competitive inhibitors of PLA2 of the invention. In addition, synergistic combinations of one of more of the non-competitive PLA2 inhibitor compounds, polymers, or compositions described here with another enzyme inhibitor are especially preferred. These include one or more cocoa-derived epicatechin polymers and orlistat, or one or more cocoa-derived epicatechin polymers and lipstatin. Additional combinations of the epicatechin-rich polymer compositions and purified polymers and combinations of them can be made with other food supplement or vitamin or herbal products, in particular but not limited to caffiene, epicatechin monomers, and/or theophylline. One of skill in the art is familiar with a multitude of healthy, natural, or bio-active supplements or compounds available in the food, nutriceutical, and pharmaceutical fields that can be used in any of the above or other listed combinations in this specification.

The compounds and compositions derived from cocoa sources, such as cocoa powders and extracts of Theobroma cacao plants and beans, may have particular effectiveness in fatty acid uptake inhibition. The epicatechin polymers found in cocoa are primarily B-type procyanidins, with some A-type procyanidins. A number of possible permutations in the polymerization of (−)-epicatechin are known.

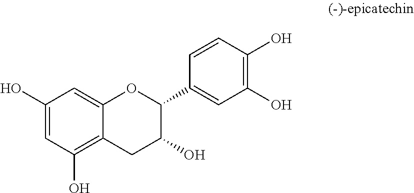 [SMILES: Oc1cc(O)c2c(c1)O[C@H](c1ccc(O)c(O)c1)[C@H](O)C2]

B-Type Polymers of Epicatechin

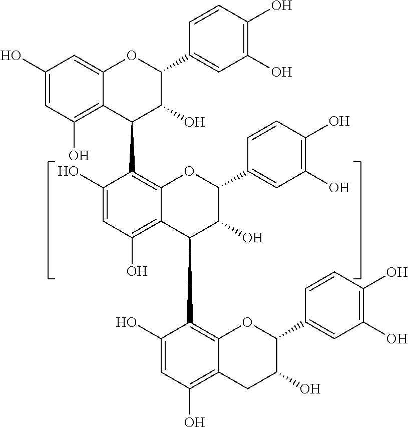

In contrast, tea and green tea are particularly rich in catechins, of which epigallocatechin gallate (EGCG) is the most abundant. Accordingly, in some embodiments of the invention a cocoa-derived polymer, purified extract, or polymer composition can be important. In other, preferred embodiments, epicatechin-rich polymers or compositions are derived only from cocoa sources or cocoa beans or cocoa bean nibs.

It is one object of the invention to provide methods of selecting and/or processing cocoa beans for producing cocoa ingredients, extracts, and compositions having enhanced levels of beneficial epicatechin-rich polymers. It is a further object of the invention to provide cocoa ingredients, including chocolate liquor, cocoa powder, low fat cocoa powder, defatted cocoa powder, and cocoa extracts having enhanced levels of epicatechin polymers and food products containing or made from these cocoa ingredients, extracts, and compositions.

In one aspect, the cocoa beans used to produce comestible products or ingredients as described here comprise more than 10% unfermented cacao beans or more than 10% raw (unfermented and un-roasted, or “Lavado”) cacao beans. The selected beans can be made into a number of cocoa compositions, such as cocoa liquor, cocoa powder, low fat cocoa powder, defatted cocoa powder, and a cocoa extract. The beans can also be roasted or treated with alkali—“Dutched” as known in the art. There are numerous food or beverage products one could make from the cocoa-derived epicatechin polymer compositions of the invention, including but not limited to a chocolate product, a milk chocolate product, a dark chocolate product, a semisweet or bittersweet chocolate product, a chocolate-flavored product, a chocolate confectionery, a chocolate-flavored confectionery, a beverage, a chocolate beverage, a chocolate-flavored beverage, a dietary supplement, a chocolate-coated product, a low fat chocolate product, a baked chocolate product, such as a cake, brownie, or bread product, or a low-sugar chocolate product.

Specific cocoa-derived compounds or compositions comprising several cocoa-derived epicatechin polymers can, in some embodiments of the invention, be specific or substantially specific for inhibiting phospholipase activity, such as phospholipase A₂ (PLA2) activity. For example, in some preferred embodiments, inhibitor compositions of the present invention do not inhibit or do not significantly inhibit or essentially do not inhibit other lipases, such as pancreatic triglyceride lipase (PTL) and carboxyl ester lipase (CEL), or amylases. In some preferred embodiments, inhibitor compositions of the present invention inhibit PLA2, and preferably phospholipase-A2 IB, but do not inhibit or do not significantly inhibit or essentially do not inhibit PLA1 and/or PLB. In some embodiments, the phospholipase inhibitor compositions preferably act on the gastrointestinal mucosa and significantly inhibit or essentially inhibit membrane-bound phospholipases there. In some embodiments, inhibitor compositions of the present invention, or specific epicatechin polymers, inhibit activity of PLA2 by interacting with its catalytic site.

While the Examples below show levels of inhibition, the complete or irreversible inhibition of PLA2 or pancreatic lipases is not critical to the invention. Thus, the term “inhibits” and its grammatical variations are not intended to require a complete inhibition of enzymatic activity. For example, it can refer to a reduction in enzymatic activity present in a sample of the duodenum small intestine during digestion, or in some other specifically-stated condition, by at least about 30%, preferably at least about 50%, at least about 75%, preferably by at least about 90%, more preferably at least about 98%, and even more preferably at least about 99% of the activity of the enzyme in the absence of the inhibitor. Most preferably, it refers to a reduction in enzyme activity with an effective amount sufficient to produce a therapeutic and/or a prophylactic benefit in at least one condition being treated in a subject. Conversely, the phrase “does not inhibit” or “essentially does not inhibit” and its grammatical variations does not require a complete lack of inhibitory effect on the enzymatic activity. For example, it refers to situations where there is less than about 5%, preferably less than about 2%, and more preferably less than about 1% of reduction in enzyme activity. Most preferably, it refers to a reduction in enzyme activity so that a measurable effect cannot be observed.

Without limiting the scope of the invention to any particular hypothesis or method of action, the benefits of the cocoa-derived and epicatechin polymers can be the result of one or more of a number of effects brought about by reduced PLA2 activity or the effect on the proteins associated with inflammatory responses in a cell. For example, inhibition of PLA2 activity may reduce transport of phospholipids through the gastrointestinal lumen, or through the small intestine apical membrane, causing a depletion of the pool of phospholipids (e.g. phosphatidylcholine) in enterocytes. This may be the case in mammals fed a high fat diet. In such cases, the de novo synthesis of phospholipids may not be sufficient to sustain the high turnover of phospholipids, e.g. phosphatidylcholine, needed to carry the triglycerides in chylomicrons. In other aspects, plasma levels of the cocoa-derived polymers influence or directly effect the protein levels in cells and tissue of the body, especially adipose cells and in particular visceral adipose tissue.

The phospholipase inhibitors useful in the present invention, or pharmaceutically acceptable variants or salts thereof, can be delivered to a subject using a number of routes or modes of administration. Preferably, the inhibitor compositions are delivered orally or as part of a chocolate or cocoa product, such as one or more as described in B. Minifie, Chocolate, Cocoa, and Confectionery, 3d Ed., Aspen Publishers. The term “pharmaceutically acceptable variants or salts” means those variant compounds and salts that retain the biological effectiveness and properties of the polymer compounds of the present invention, and which are not biologically or otherwise undesirable. The phospholipase inhibitors (or pharmaceutically acceptable variants or salts thereof) may be administered alone or in the form of a pharmaceutical composition where the active compound(s) is in admixture or mixed with one or more pharmaceutically acceptable carriers, excipients, or diluents known or available in the art. Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen. A variety of pharmaceutical compositions can be prepared by methods known or apparent to those skilled in the art and are described in more detail in, for example, Remington's Pharmaceutical Science, 17th ed., Mack Publishing Company, Easton, Pa. (1985), and the later 18^{th} and 19^{th} editions, which are all incorporated herein by reference.

In preferred embodiments, the invention comprises method to inhibit fatty acid uptake in an animal by administering a comestible composition containing an effective amount of cocoa-derived epicatechin polymers having from 2-14 epicatechin units or a cocoa-derived epicatechin polymer of 2 or more epicatechin units. The effect of the administration is a measureable or effective reduction in pancreatic lipase A2 or pancreatic lipase enzyme activity, preferably by 50% or more of the normal, untreated activity in the animal, or the untreated activity present in a sample of digestive fluids in the duodenum or stomach of the animal. In another aspect, various ranges or specific subsets of cocoa-derived or epicatechin-rich polymers can be selected and used, including any one or more of the polymers of 2 to 14 units (DP=2-14, where DP refers to degree of polymerization), polymers from 5-13 units (DP 5-13), or polymers of 5 or more units (DP>5) or 2 or more units (DP>2). The Examples below specifically refer to DP5, DP7, DP8, DP10, and DP2-DP10, and DP7+ polymer compositions, any which can be selected or used in combination.

The methods also include incorporating the comestible composition into a cocoa or chocolate food or ingredient, such as chocolate liquor, a cocoa powder, or a cocoa bean extract. Additional cocoa-derived epicatechin polymers can be added to the composition beyond those present in the native cocoa powder, for example. A comestible composition can thus include cocoa butter, where the inhibitors present effectively reduce the uptake of fatty acids found in cocoa butter. Accordingly, chocolate products having reduced fatty acids “availability,” as measured by the actual amount of fatty acids passing into the bloodstream from the gut, are possible.

The invention also includes methods where the cocoa-derived epicatechin polymers or the compositions of them act as a competitive inhibitor of pancreatic lipase A2 enzyme activity, specifically.

Various cocoa bean samples or products can be used as a source of the polymer compositions of the invention. In a preferred example, the cocoa-derived polymers are derived from unfermented cocoa beans, or raw “Lavado” beans. Examples with “Regular” beans, those that have been fermented and roasted in conventional processes know in the art, can also be used. As noted above, “Dutched” samples have been treated with alkali, as known in the art.

In addition, the invention provides a cocoa ingredient selected from chocolate liquor, cocoa powder, cocoa extract, low fat cocoa powder, defatted cocoa powder, and non fat cocoa solids, wherein the level of cocoa-derived epicatechin polymers having from 2-14 polymer units present exceeds 50 ug/mg, or 100 ug/mg, or 200 ug/mg, or 500 ug/mg or more. Certain examples for the dosage for human oral delivery can vary, but examples include 5-80 mg, or 600 mg/day or more.

Throughout this disclosure, applicants refer to texts, journal articles, patent documents, published references, web pages, and other sources of information. One skilled in the art can use the entire contents of any of the cited sources of information to make and use aspects of this invention. In particular, the article by Gu et al. (2011) J. Agric. Food Chem. 59(10):5305-5311, is incorporated herein by reference. Each and every cited source of information is specifically incorporated herein by reference in its entirety. Portions of these sources may be included in this document as allowed or required. However, the meaning of any term or phrase specifically defined or explained in this disclosure shall not be modified by the content of any of the sources. The description and examples herein are merely exemplary of the scope of this invention and content of this disclosure and do not limit the scope of the invention. In fact, one skilled in the art can devise and construct numerous modifications to the examples listed below without departing from the scope of this invention.

### DETAILED DESCRIPTION OF EXEMPLARY EMBODIMENTS

In one aspect the invention involves the use of cocoa beans from any source, and products made from them or derived from them. The terms “cocoa extract,” “cocoa bean composition,” and “cacao bean composition” can be any of a variety of products and combinations of the cocoa bean-derived products noted in this disclosure. “Cocoa bean composition,” “cacao bean composition” and “cocoa products” are essentially interchangeable and mean a product made from a cacao bean. A “cacao bean sample” or a “cocoa bean sample” is a collection of cacao beans or the nibs of such beans from a desired set of sources or set of processing conditions. In addition, combinations of cocoa products or cocoa extracts involving cacao beans treated, processed, or selected under conventional methods can be combined with cacao bean compositions of the invention. These compositions and extracts can be used in any cocoa ingredient, which in turn can be used in any composition or product for human consumption, including foods, confections, beverages, and supplements.

Cocoa powder, as understood in the art, contains approximately 10-25% lipid fraction (cocoa butter). However, all or a percentage of the fat can be removed from the powders by pressing, by solvent or supercritical solvent extraction or any number of other methods, as known in the art. Thus, natural, defatted and/or low fat or non-fat cocoa powders are specifically included in the cocoa products or ingredients described here. Other cocoa products, such as breakfast cocoa, cocoa extracts, and chocolate liquor can also be produced from the invention.

In some embodiments, the recommended dosage of a phospholipase inhibitor is between about 0.1 mg/kg/day and about 1,000 mg/kg/day, or in other embodiments about 100 to about 1,000 mg/day. The effective amount for use in humans can be determined from animal models. For example, a dose for humans can be formulated to achieve circulating and/or gastrointestinal concentrations that have been found to be effective in animals, e.g. a mouse model. A person of ordinary skill in the art can determine phospholipase inhibition by measuring the amount of a product of a phospholipase, such as lysophosphatidylcholine (LPC), a product of PLA2. The amount of LPC can be determined, for example, by measuring small intestine, lymphatic, and/or postprandial serum levels. Another technique for determining the amount of phospholipase inhibition involves taking direct fluid samples from the gastrointestinal tract. A person of ordinary skill in the art would also be able to monitor in a subject the effect of a phospholipase inhibitor of the present invention, such as by monitoring cholesterol and/or triglyceride serum levels. Other techniques would be apparent to one of ordinary skill in the art. Other approaches for measuring phospholipase inhibition and/or for demonstrating the effects of phospholipase inhibitors of some embodiments are further illustrated in the examples below.

The cocoa compositions and products of the present invention can contain enhanced levels of epicatechin polymers or polymer compositions by supplementing or adding to levels from a purified source or extract.

The present invention also includes food products containing cocoa ingredients having enhanced levels of epicatechin-rich polymers or compositions. The term “food product” includes any edible or consumable product that can be ingested by humans or animals to provide nourishment or provide supplements, and includes but is not limited to chocolate foods, chocolate bars, chocolate candies, steeped cocoa beverages, chocolate drinks, chocolate-flavored foods, chocolate-flavored bars, chocolate-flavored candies, chocolate-flavored drinks, chocolate-coated foods, chocolate-coated bars, chocolate-coated candies, milk chocolate, dark chocolate, baking chocolate, semi-sweet baking chips, baked chocolate products, such as cakes, brownies and breads, reduced-sugar chocolate and reduced-fat chocolate.

In another aspect, the invention includes ingredients or compositions, including pharmaceutical compositions, having natural epicatechin-rich polymer compounds derived from Theobroma cacao, which compounds may include one or more of the DP 2-14 polymers or other polymer compositions referred to herein.

### EXAMPLES

Cocoa epicatechin polymers or cocoa-derived procyanidins from dimers to decamers (degree of polymerization=2 to 10) are prepared from one of three cocoa bean extracts (regular, lavado, and dutched). Both the polymer compositions and the extracts can be used in the analysis as described here. The extracts can be prepared by first defatting a cocoa sample with hexane, which involves mixing samples with hexane. The samples are centrifuged, the hexane poured off, and the residue allowed to dry overnight. Dry residue is extracted with 70/30/05 (v/v/v) acetone/water/acetic acid while shaking for 30 minutes This solution can be filtered through Whatman #4 filter paper, or the equivalent. The solvent is removed by placing under vacuum or in hood overnight, and the resulting extract can be used directly for studies. Stock solutions can be prepared by dissolving the cocoa samples in DMSO (EMD Chemicals Inc.; Gibbstown, N.J.). Tests of purities level of all cocoa procyanidin compositions show more than 85% purity by HPLC. Standards for (−)Epicatechin (EC) was purchased from Sigma Chemical Co. (St. Louis, Mo.), and Orlistat (Xenical, Alli) was purchased from Sigma Chemical Company. Analysis of HPLC purified peaks can be performed using MALDI mass spectrometry with a sodium adduct. Typical results for various cocoa polymer m/z are DP=2 at 601; DP=3 at 889; DP=4 at 1,177; DP=5 at 1,465; DP=6 at 1,753; DP=7 at 2,041; DP=8 at 2,329; DP=9 at 2,617; and DP=10 at 2,907. These values are consistent with the polymers being epicatechin polymers of the stated degree of polymerization.

Measurement of Enzyme Activities In Vitro:

The activities of pancreatic α-amylase, pancreatic lipase and phosoholipase A2 are measured by in vitro inhibition assays and are expressed as percentage of control (blank). α-Amylase can be purchased from porcine pancreas and Red-Starch can be purchased from Megazyme (Wicklow, Ireland). Lipase can be purchased from porcine pancreas (Type II) and 4-Nithophenyl butyrate (4-NPB, 98%) can be purchased from Sigma-Aldrich (St. Louis, Mo.). EnzChek® Phospholipase A2 Assay Kit can be purchased from Invitrogen (Carlsbad, Calif.) and Molecular Probes Inc. (Eugene, Oreg.). All the other reagents are of analytical grade. The dose-response curves are constructed by plotting enzyme activity (% control) against a range of concentrations of cocoa procyanidins and cocoa extracts. The inhibitory constant 50% (IC50) of each cocoa procyanidin and extract is determined by interpolation or extrapolation of a dose-response curve using GraphPad Prism software (San Diego, Calif.). The concentrations of cocoa procyanidins and cocoa extracts are tested at 0-100 uM and 0-200 ug/ml, respectively.

Pancreatic α-Amylase Inhibition Assay

The pancreatic α-Amylase (PA) inhibition assay is performed using the chromogenic method adapted from Megazyme. Red-starch is used as the substrate, which is starch dyed with Procion Red MX-5B. Pancreatic α-amylase solution is prepared by dissolving in 20 mM phosphate buffer (pH 6.9) containing 6.7 mM sodium chloride. After pre-incubation in water bath at 37° C. for 5 min, red-starch in potassium chloride solution and buffered α-amylase solution are combined with cocoa procyanidins/cocoa extracts or control (distilled water). On incubation of the mixture at 37° C. for 10 min, the red-starch is depolymerised to produce low molecular weight of red dyed fragments, and the reaction is stopped by addition of 95% ethanol. After equilibrating to room temperature, the high molecular weight material is removed by centrifugation. The supernatant is transferred to cuvettes and its absorbance is measured at 510 nm using a spectrophotometer (BECKMAN DU® 650).

Pancreatic Lipase Inhibition Assay

The measurement of pancreatic lipase (PL) activity involves the cleavage of 4-nitrophenylbutyrate (4-NPB) to release butyric acid and 4-nitrophenol (4-NP). The liberation of 4-NP results in a color change that can be monitored at 400 nm spectrophotometerly. The cocoa procyanidins or cocoa extracts were combined with pancreatic lipase and 0.1 M Tris-HCI buffer (pH 8), and then 4-NPB is added to start the reaction. Following incubation at room temperature for 10 min, absorbance is read at 400 nm. Orlistat, a clinically-used inhibitor of PL, is used as a positive control.

Phospholipase A2 Inhibition Assay

The phospholipase A2 (PLA2) activity is measured using a fluorometric method with a Phospholipase A2 Assay Kit. The substrate Red/Green BODIPY® PC-A2 is a glycerophosphocholine dye-labeled with BODIPY® at sn-1 and sn-2. The phospholipase A2 hydrolyzes the sn-2 ester of phospholipid to release a lysophospholipid and a fluorophore. Buffered PLA2 solution (pH 8.9) and cocoa procyanidins or cocoa extracts are added to the individual wells of a 96-well microplate. An aliquot of PLA2 substrate is dispensed to each microplate well to start the reaction. After incubation at room temperature (protected from light) for 10 min, the PLA2 activity is determined by measuring its fluorescence intensity (Fluoroskan Ascent FL, from ThermoFisher Scientific Inc.) at excitation and emission wavelengths equal to 485 nm and 538 nm, respectively.

Kinetic Analysis

Cocoa procyanidin pentamer (DP=5) and decamer (DP=10) as well as regular cocoa extract are selected to perform the kinetic analysis of inhibition against pancreatic lipase (2.2.2) and phospholipase A2 (2.2.3). Cocoa procyanidins or cocoa extracts are held at constant concentrations and incubated in the presence of increasing concentrations of substrates together with enzymes and buffer solutions. Enzyme activities are determined as described above (2.2). These data were used to construct Michaelis-Menten plots by GraphPad Prism software (San Diego, Calif.), and the Vmax (maximum velocity) and Km (Mechaelis-Menten constant, concentration of substrate that produces half-maximal velocity) are assessed. Vmax and Km values of each enzyme in the presence of inhibitors (cocoa procyanidin pentamer, decamer and regular cocoa extract) are analyzed by one-way ANOVA or Student's t-test, and the mode of inhibition (competitive, non-competitive, or mixed-type inhibition) of each inhibitor was determined accordingly.

Statistical Analysis

Data are expressed as mean±standard deviation (SD) of the mean of at least three independent experiments. P-values lower than 0.05 are considered as statistically significant. All statistical analyses are performed using GraphPad Prism software (San Diego, Calif.).

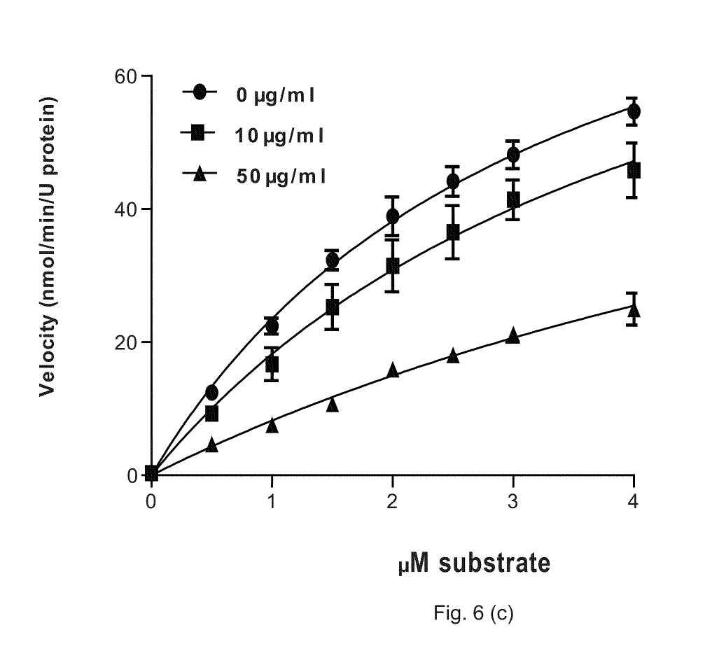

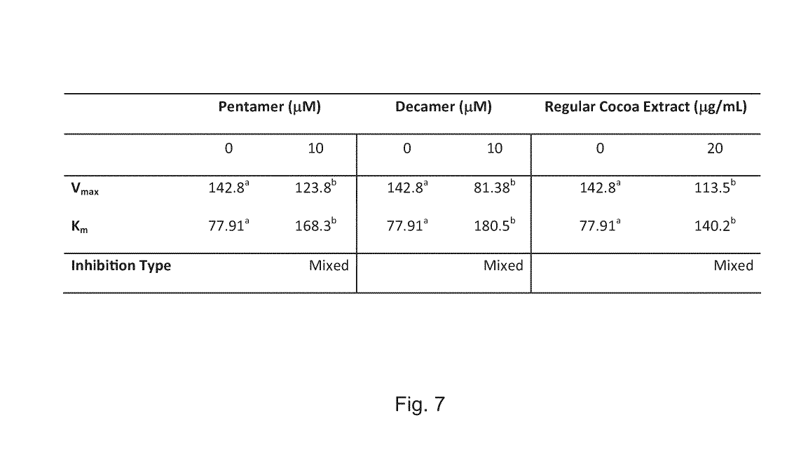

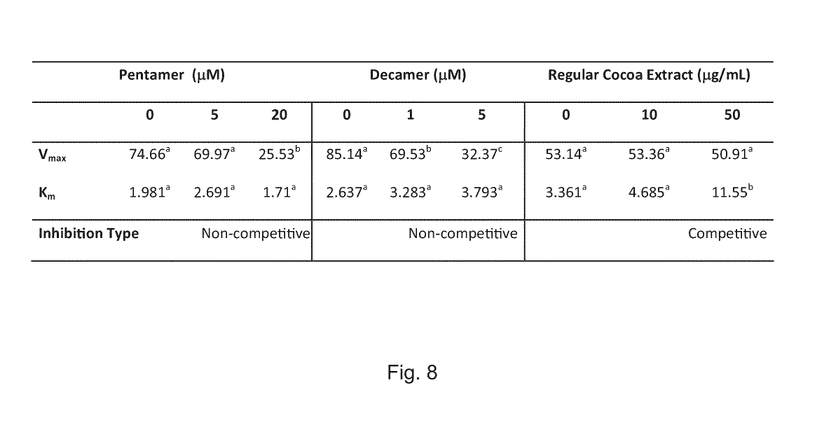

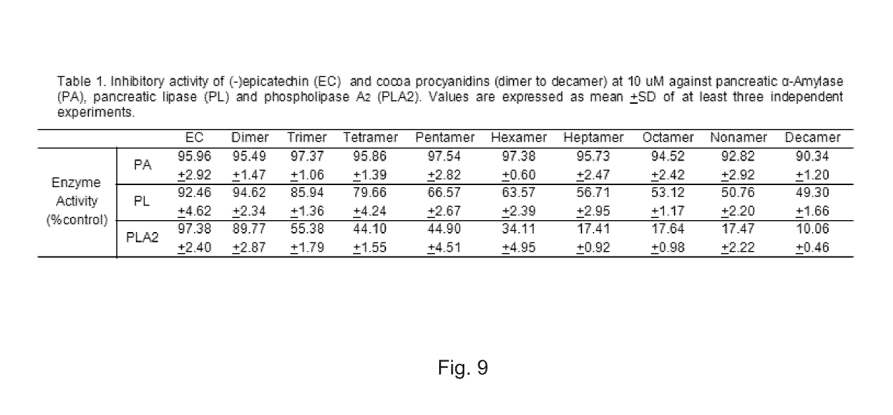

Table 1 of FIG. 9 shows the high inhibitory activity of EC polymers on PLA2 activity in particular. Inhibition increases dramatically from DP 7 to DP 10.

FIG. 7 includes a Table showing the Vmax and Km of pancreatic lipase in the absence and presence of various concentrations of cocoa procyanidin pentamer, decamer, and the regular cocoa extract. FIG. 8 includes a Table showing the Vmax and Km of phospholipase A2 in the absence and presence of various concentrations of cocoa procyanidin pentamer, decamer, and the regular cocoa extract. The type of inhibition that occurs based upon this analysis is also listed in each Table. As indicated, both the pentamer and decamer compounds non-competitively inhibit PLA2

Competitive inhibition indicates that the inhibitor competes with the substrate for access to the active site of the enzyme. Such inhibition can be overcome if sufficient amount of substrate is present to out-compete the inhibitor. By contrast, a non-competitive inhibitor binds to a site on the enzyme other than the active site. This binding results in a conformational change that makes the enzyme less active. Non-competitive inhibition cannot be overcome by addition of more substrate. Mixed-type inhibition means that an inhibitor exhibits characteristics of both a competitive and non-competitive inhibitor. For purposes of the methods of the invention to treat animals or humans or inhibit fatty acid and/or triglyceride uptake in animals or humans, the non-competitive inhibition demonstrated by the purified cocoa epicatechin polymers is especially advantageous.

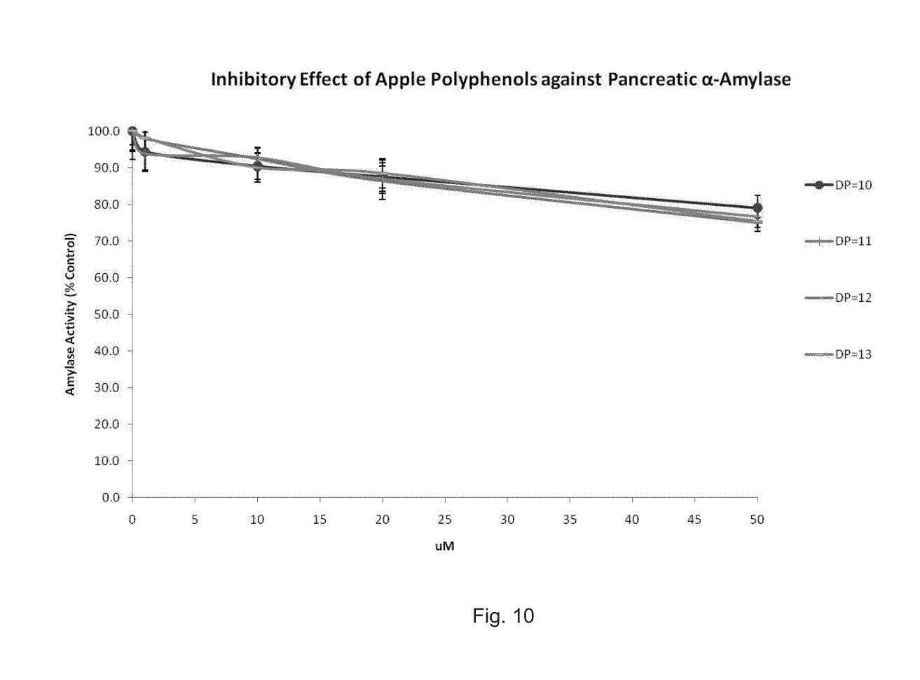

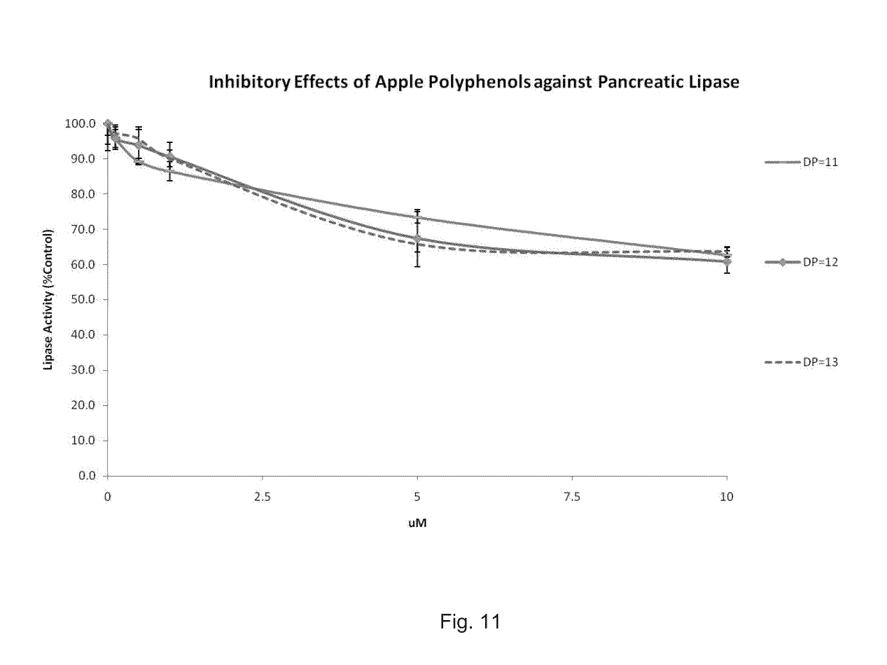

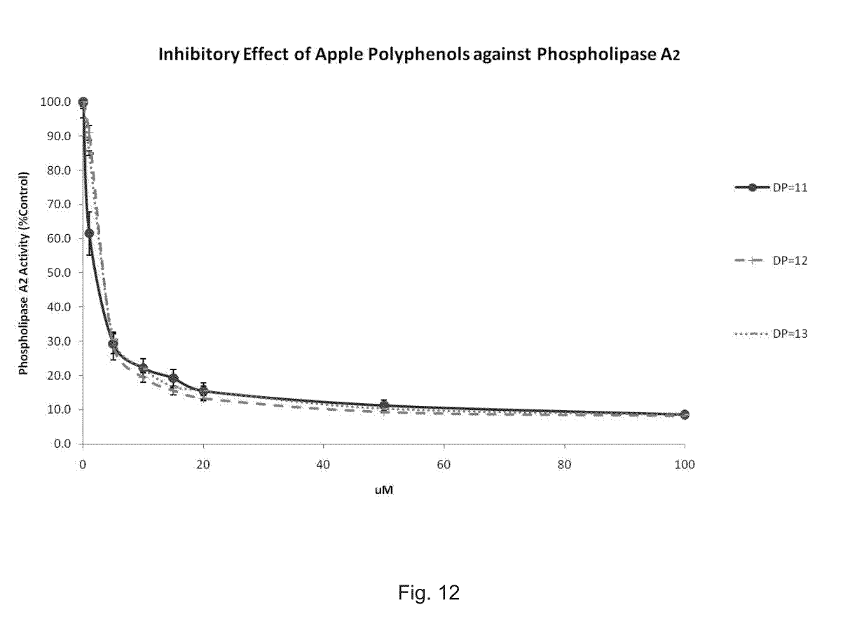

As shown in FIGS. 10, 11 and 12, higher procyanidin polymers derived from plants inhibit α-amylase, pancreatic lipase, and phospholipase A2 in a similar manner as shown in the results above. The data in these Figures include the use of polymers DP 10 to DP 13. As is the case for cocoa-derived compounds and compositions, these compounds are strong inhibitors of PLA2 activity. Accordingly, the procyanidin polymers that can be used in the products, compositions, and methods of the invention include polymers of two or more epicatechin units.

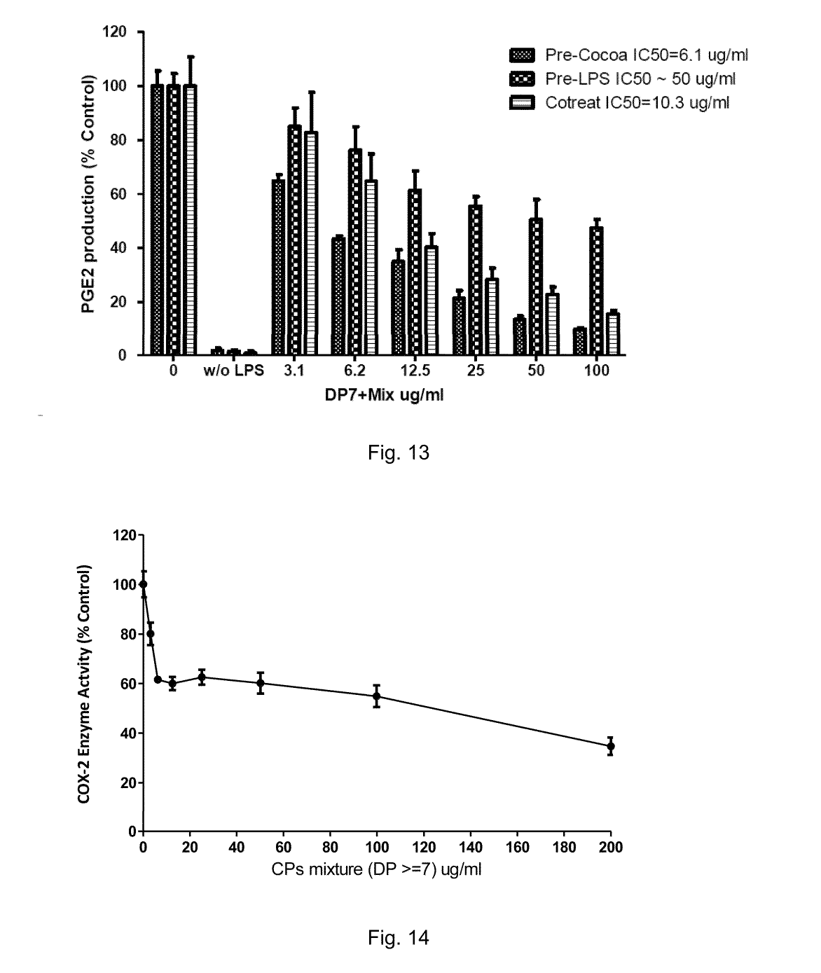

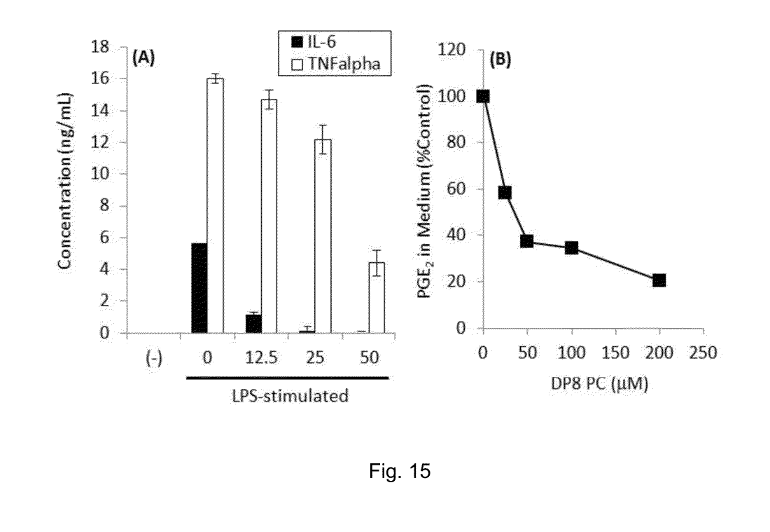

FIGS. 13 and 15 show the effect of epicatechin-rich and cocoa-derived polymer compositions and purified polymers on cultured mammalian cells that are routinely used in the assay for compounds that interact with the production of compounds involved in inflammatory processes in mammal and humans. These cells produce both inflammatory eicosanoids and inflammatory cytokines when stimulated. FIG. 13 shows the effect of high molecular weight polymer mixture (degree of polymerization 7 or greater; noted as DP7+Mix in the graph) on the production of the inflammatory eicosanoid prostaglandin (PG)E₂ by lipopolysaccharide (LPS, 1 μg/mL)-stimulated RAW264.7 macrophage cells. Three treatment regimens are shown: (a) Pre-treatment with DP7+ for 6 h then stimulation with LPS for 6 h (Pre-cocoa); Pre-stimulation with LPS for 6 h then treatment with DP7+ for 6 h (Pre-LPS); and Co-treatment with DP7+ and LPS for 12 h (Cotreat). All three treatment regimens reduced the production of PGE₂ compared to control (0) and in a dose-dependent manner. Accordingly, administration of epicatechin-rich polymer compositions can be used to reduce inflammatory reactions in mammals and humans.

FIG. 14 shows the inhibitory effect of the DP7+ mixture of cocoa-derived polymers on purified cyclooxygenase 2 (COX-2) enzyme activity. Mean inhibitory concentration (IC₅₀) is 58 ug/ml. As the COX-2 enzyme is directly involved in the production of inflammatory eicosanoids, the inhibition by epicatechin-rich polymers indicates that these polymers can act at the level of inhibiting COX-2 enzyme in mammals and humans, similar to other orally administered COX-2 inhibitors, as well as the level of PLA2 enzyme. The potential for dual inhibition of at least these two enzymes integral to eicosanoid biosynthesis can provide synergistic or especially effective treatments to reduce the level of eicosanoids produced by a cell or by certain tissues, either acutely, temporarily, or over a period of time, with administration regimens and dosages.

FIG. 15 depicts additional data on mammalian cell cultures. Graph (A) at left shows the inhibition of the production of inflammatory cytokines IL-6 and TNF in LPS stimulated macrophages by administration of purified polymer DP=8 composition. The graph (B) at right shows the inhibition of PGE₂ production in LPS stimulated macrophages by purified DP=8 polymer composition. In these assays, macrophage cell cultures (RAW264.7) are pre-treated with DP=8 octamer composition for 10 h and then stimulated with LPS for 12 h and assayed. As above in FIG. 13, these data show that administration of a specific epicatechin-rich polymer DP=8 can be used to reduce inflammatory reactions in mammals and humans.

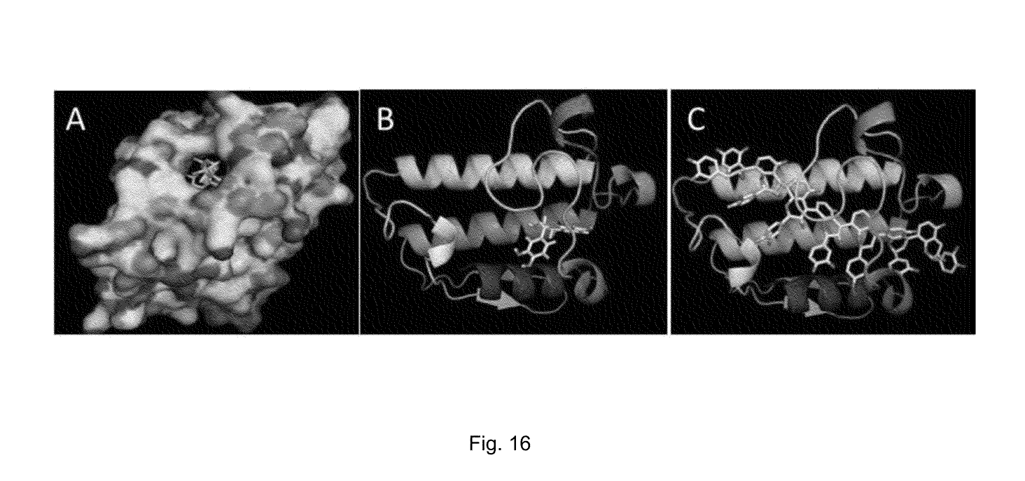

FIG. 16 depicts the binding interactions of PLA₂ enzyme. Panel A shows ball-and-stick substrate bound in tunnel region of space-filling model of PLA₂ enzyme. Panel B depicts DP=2 polymer interacting with secondary and tertiary structures of PLA₂ enzyme, including binding to helices that form tunnel walls as shown in Panel A. Panel C depicts binding of DP=7 to secondary and tertiary structures of PLA₂ enzyme. In Panel C, additional binding sites of DP=7 polymer exhibits potential for increased potency as inhibitory molecule.

Specific Effects on Eicosanoids and Arachidonic Acid (AA) Metabolism

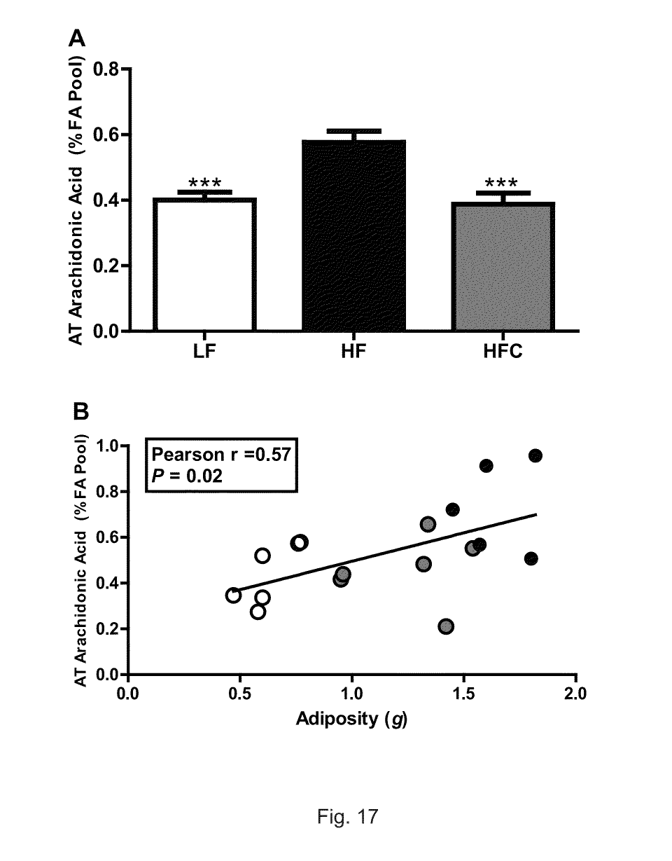

Eicosanoids represent a group of inflammatory lipid mediators derived from adipose tissue. Arachidonic Acid (AA) is a ω-6 fatty acid and is the precursor of various eicosanoids. FIG. 17 exemplifies the impact of cocoa supplementation on arachidonic acid levels in adipose tissue (AT) (FIG. 17A) and its correlation with adiposity (FIG. 17B). Arachidonic acid levels can be determined in duplicate from a set of representative retroperitoneal adipose tissue samples with LF (low fat diet), n=6; HF (high fat diet), n=5; and HFC (high fat+cocoa), n=6, where diet is maintained for 16 to 18 weeks during study. Retroperitoneal adipose tissue represents the visceral adipose tissue implicated in inflammatory and other adverse health conditions, as opposed to epidural adipose tissue. Values expressed as a mean±SEM in the Figures. The mean can be compared by one-way ANOVA with Dunnett's post-test (HF as control). *** P<0.001. A correlation between arachidonic acid levels and the adiposity was assessed by GraphPad Prism 5.0 (San Diego, Calif.). Cocoa-supplemented mice show a 32.8% reduction in AA levels in adipose tissue compared to HF (high fat diet) group (P<0.001, FIG. 17A). Moreover, AA levels in adipose tissue is positively correlated with adiposity (Pearson r=0.57, P=0.02) as shown in FIG. 17B.

Effects on Markers for Inflammatory Conditions

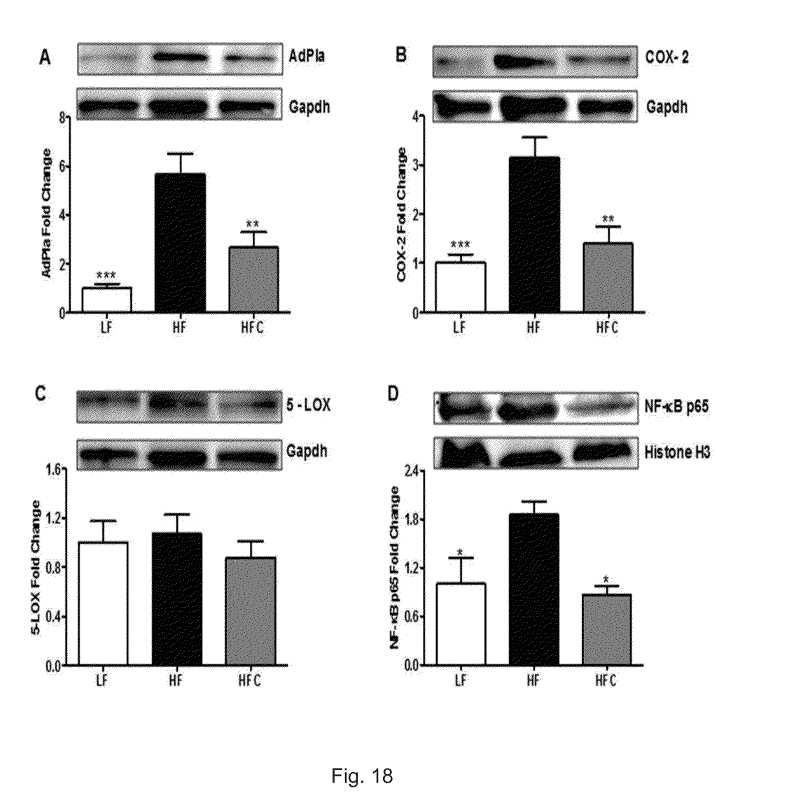

In adipose tissue, AA is mainly released from the membrane phospholipids by the action of AdPla, and then AA can be further converted to eicosanoids by the COX enzymes and/or the LOX enzymes. Western blot results (FIG. 18 A-C) show that the protein expression of AdPla and COX-2 were reduced by nearly 50% in the cocoa treated group (P<0.01). By contrast, there is no significant effect of cocoa on the expression of 5-LOX among the three groups. Thus, cocoa and cocoa procyanidins appear to preferentially influence the prostaglandin pathway, which is linked to inflammation responses. Furthermore, the expression of NF-κB (p65 subunit) in nucleus can be determined (FIG. 18 D). Cocoa supplementation significantly decreases NF-κB p65 expression in the nucleus compared to HF (high fat) control group (P<0.05), which may reduce its activation resulting in down-regulation of inflammatory gene expression.

FIG. 18 shows the effect of cocoa supplementation on the protein expression of (A) AdPLA, (B) COX-2, (C) 5-LOX and (D) nuclear NF-κB p65 in adipose tissue of C57bl6/J mice. Protein expression of eicosanoid-generating enzymes (AdPLA, COX-2 and 5-LOX) was determined in whole cell lysate from a set of representative mouse retroperitoneal adipose tissue samples with n=6 for each group. Protein expression of NF-κB p65 can be measured in nuclear fractions from a set of representative mouse retroperitoneal adipose tissue samples with n=6 for each group. Values can then be expressed as mean±SEM. The mean can be compared by one-way ANOVA with Dunnett's post-test (HF as control). * P<0.05, ** P<0.01, *** P<0.001.

Plasma Endotoxin Levels and Improvement in Gut Barrier Function

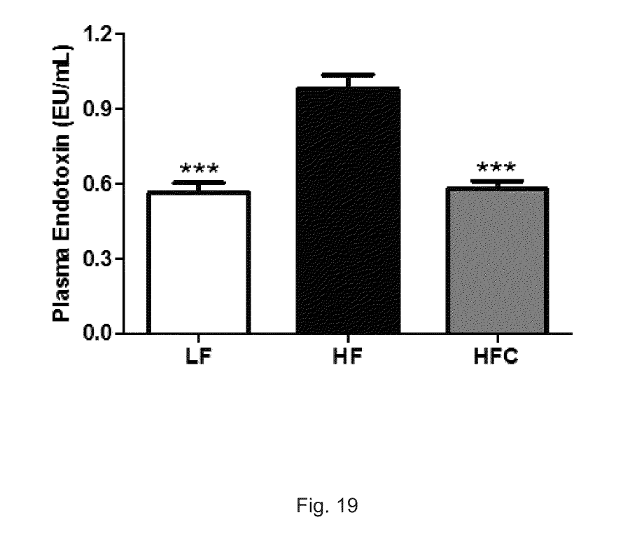

Mice (C57bl6/J mice) fed a 16 to 18 week HF (high fat) diet show a 1.8-fold increase in plasma endotoxin levels by (P<0.001) compared to LF-fed (low fat) controls (FIG. 19). Cocoa supplementation reduces the elevation of plasma endotoxins and results in 40.8% lower (P<0.001) plasma endotoxin levels compared to HF-fed mice (FIG. 19). Thus, cocoa supplementation improves the plasma endotoxin levels in animals and can therefore positively effect conditions associated with metabolic endotoxemia, such as diabetes. Plasma endotoxin levels can be determined at the end of experiment with LF (low fat) n=23; HF (high fat) n=21; and HFC (high fat+cocoa) n=24. The plasma values can be expressed as mean±SEM. The mean can be compared by one-way ANOVA with Dunnett's post-test (HF as control). *** P<0.001

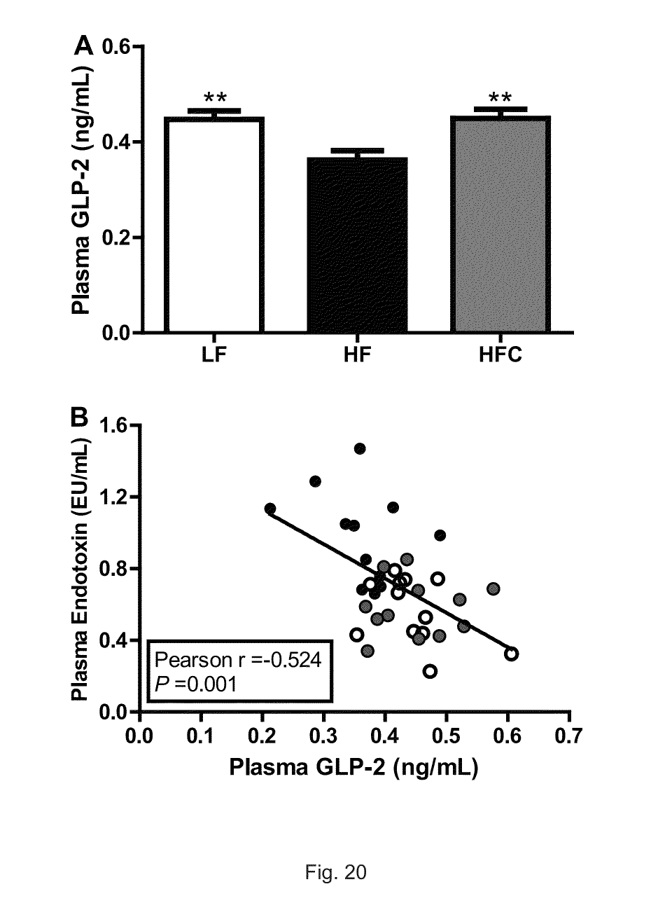

Related tests can use the endotoxin marker glucagon-like peptide-2 (GLP-2), which is a gastrointestinal hormone having a number of actions in the intestine, including stimulation of mucosal growth, improvement of gut barrier function, and reduction of intestinal permeability. Compared to LF-fed mice, obese mice fed with a HF diet had lower levels of plasma GLP-2 (P<0.01), and cocoa treatment increased GLP-2 levels by 36.1% (P<0.01) compared to HF-fed mice (FIG. 20 A). Moreover, plasma GLP-2 levels had a strong negative correlation between the plasma endotoxin levels (Pearson r=−0.52, P=0.001), which evidences a role for GLP-2 in metabolic endotoxemia (FIG. 20 B). FIG. 20 shows impact of cocoa supplementation on Plasma GLP-2 levels (FIG. 21A) and its correlation with plasma endotoxin levels in C57bl6/J mice (FIG. 20 B). Plasma GLP-2 levels can be determined at the end of experiment using a set of representative plasma samples with n=12 for each group. Values are expressed as mean±SEM. Means can be compared by one-way ANOVA with Dunnett's post-test (HF as control). *** P<0.001. The correlation between plasma GLP-2 levels and plasma endotoxin levels can be assessed by GraphPad Prism 5.0 (San Diego, Calif.).

Combined and individually, the examples and results here support the beneficial chronic use of cocoa powder and cocoa procyanidins in improving various health conditions when administered orally. Adipose tissue has an important endocrine function in the regulation of whole-body metabolism. Obesity leads to a chronic inflammation of the adipose, which disrupts this endocrine function and results in metabolic derangements, such as type-2 diabetes and cardiovascular diseases. Bioactive food components, such as cocoa polyphenols, have been shown to suppress both systemic and adipose inflammation and have the potential to improve these obesity-associated metabolic disorders. Here, we provide evidence for the preventive effects of a long-term supplementation with dietary cocoa on adipose tissue inflammation with both in vitro and in vivo examples. While dietary cocoa supplementation for 16 or 18 weeks may not decrease the final body weight in HF-fed mice in basic dietary monitoring studies with cocoa powder, markers of hyperinsulinemia and hyperlipidemia can be significantly improved by cocoa powder treatment. Dietary supplementation with 8% (w/w) cocoa powder attributes approximately 0.6% cocoa polyphenols or about 50 mg polyphenols/kg body weight (based on the assumption that a HFC-fed mouse consumes about 3 g per day and weighs about 35 g on average).

The inventors' related work (published in Gu, et al., Eur. J. Nutr., 2013 “Dietary cocoa ameliorates obesity-related inflammation in high fat-fed mice,” doi:10.1007/s00394-103-105-1), which is specifically incorporated herein by reference, explains possible dosing regimens and descriptions of cocoa powder content for exemplary cocoa powder, preferably natural cocoa powder, that can be used in animals. This document also refers to specific effects on liver diseases and markers for liver function as well as effects on diabetes. Accordingly, the invention employing the cocoa-derived polymers and compositions here can be used for treatments and prophylactic procedures and methods for preventing and treating a number of human diseases and conditions, including liver disease, diabetes, conditions associated with metabolic endotoxemia, cardiovascular disease, insulin resistance, and inflammatory diseases or conditions associated with chronic inflammation.

In addition, the above studies show that cocoa extracts demonstrate potent inhibitory activities against key digestive enzymes in vitro, and cocoa supplementation can significantly increase fecal lipids as well as modulate systemic circulation of inflammatory cytokines (e.g. IL-6) and adiponectin in HF-fed obese mice. Thus, the effects of dietary cocoa on insulin resistance, metabolic endotoxemia, and plasma lipids shown here may be due to the inhibition of lipid absorption and modulation of cytokine secretion with cocoa and cocoa procyanidin treatments.

The examples presented above and the contents of the application define and describe examples of the many cocoa compositions, products, and methods that can be produced or used according to the invention. None of the examples and no part of the description should be taken as a limitation on the scope of the invention as a whole or of the meaning of the following claims.

1. A method of inhibiting fatty acid uptake in an animal comprising administering a comestible composition containing an effective amount of cocoa-derived epicatechin polymers from 2-14 units, wherein the activity of phospholipase A2 or pancreatic lipase enzyme is inhibited by 50% or more.

2. The method of claim 1, wherein the cocoa-derived epicatechin polymers are from 5-13 units.

3. The method of claim 1, wherein the comestible composition comprises a chocolate liquor, a cocoa powder, or a cocoa bean extract, and additional cocoa-derived epicatechin polymers are added to the composition.

4. The method of claim 1, wherein the cocoa-derived epicatechin polymers act as a competitive inhibitor of phospholipase A2 (PLA2) enzyme activity.

5. The method of claim 1, wherein at least one epicatechin polymer present acts as a non-competitive inhibitor of PLA2 enzyme activity.

6. The method of claim 3, wherein the additional epicatechin polymers added act as non-competitive inhibitors of PLA2

7. The method of claim 1, wherein the comestible composition further comprises cocoa butter.

8. The method of claim 1, wherein the cocoa-derived epicatechins are derived from unfermented cacao beans.

9. A cocoa ingredient selected from chocolate liquor, cocoa powder, cocoa extract, low fat cocoa powder, defatted cocoa powder, and non fat cocoa solids, wherein the level of cocoa-derived epicatechin polymers from 2-14 units present exceeds 200 ug/mg, and further comprising a PLA2 inhibitor composition or compound that is not derived from cocoa.

10. A method for administering a dietary regimen for a human comprising measuring a baseline triglyceride level in a subject having a baseline diet and, based on the baseline level and diet, recommending an effective amount of a epicatechin polymer composition to be ingested daily and with meals to reduce fatty acid uptake.

11. A composition comprising a fatty acid uptake-inhibiting amount of cocoa-derived epicatechin polymers of 2 to 10 units in polymeric length, formulated for oral administration, wherein the amount of epicatechin polymers present is capable of non-competitively inhibiting approximately 50% of the PLA2 enzyme activity present in the digestive system of an animal.

12. A combination of at least one purified non-competitive inhibitor of PLA2 selected from epicatechin polymers DP 5 to DP 14 and a purified competitive inhibitor of PLA2 or PA.

13. The combination of claim 12, further comprising one or more of tetrahydrolipstatin, phaseolamine, cetilistat, crocetin, lipstatin, vibralactone, and green tea catechins.

14. A product for oral administration comprising a cocoa derived purified extract containing type-B procyanidin polymers, wherein the type and amount of polymer present are selected so that the product contains an effective amount of inhibitor to non-competitively inhibit about 20% or more of the PLA2 enzyme present during the duodenal digestion of an average meal.

15. The product of claim 14, further comprising a competitive inhibitor of PLA2 or pancreatic lipase.

16. The product of claim 14, further comprising one or more of tetrahydrolipstatin, phaseola-mine, cetilistat, crocetin, lipstatin, vibralactone, and green tea catechins.

17. The product of claim 14, wherein a DP 5 or higher epicatechin-rich polymer is selected.

18. The product of claim 14, wherein the epicatechin-rich polymer selected is one or more of DP 2 to DP 10.

19. The product of claim 14, wherein the epicatechin-rich polymers are derived only from cocoa bean nibs.

20. (canceled)

21. (canceled)

22. A pharmaceutical composition comprising a purified a DP 5 or higher epicatechin-rich polymer derived from cocoa.

23. (canceled)

24. A method of inhibiting production of inflammatory eicosanoids in a mammalian adipose or immune cell comprising administering a comestible composition containing an effective amount of cocoa-derived or epicatechin-rich polymer composition from 2-14 units, wherein the activity of COX-2 enzyme activity is inhibited by 30% or more.

25. A method of inhibiting production of inflammatory cytokines in a mammalian adipose or immune cell comprising administering a comestible composition containing an effective amount of epicatechin-rich polymer composition from 2-14 units, wherein the production of IL-6 or TNFa is reduced by 50% in a stimulated cell.

26. A composition as claimed in claim 22, further comprising one or more of epicatechin monomers, caffeine, theobromine, or theophylline.

27. A composition as claimed in claim 22, wherein the polymer is a purified epicatechin-rich polymer composition of one or more of DP5, DP6, DP7, DP8, DP, or DP10.

28. A prophylactic method of treating an animal to reduce chronic inflammatory symptoms comprising preparing a cocoa-derived polymer composition from a cocoa powder or cocoa product, administering the composition daily to a subject to deliver the polymer equivalent of 50 or more grams of natural cocoa powder daily, wherein the cocoa-derived polymer composition is contained in an orally administrable product.

29. The method of claim 28, further comprising monitoring the plasma endotoxin levels of the subject after administration.

30. The method of claim 28, further comprising monitoring the plasma GLP-2 levels of the subject after administration.

31. A product as claimed in claim 14, further comprising one or more of epicatechin monomers, caffeine, theobromine, or theophylline.

32. A product as claimed in claim 14, wherein the extract consists essentially of a purified epicatechin-rich polymer composition of one or more of DP5, DP6, DP7, DP8, DP, or DP10.
